(19) United States
(12) Patent Application Publication
(10) Pub. No.: US 2024/0245815 A1
(43) Pub. Date: Jul. 25, 2024

(54) Plasma Module, Disinfection and Sterilization Apparatus, and Air Duct Assembly

(71) Applicants: Donglei WANG, Zhuhai (CN); Crawford Global Limited, Hong Kong (CN); Dernier and Hamlyn Inc., Wheeling, IL (US); ETI Solid State Lighting Inc, Wheeling, IL (US)

(72) Inventors: Donglei WANG, Zhuhai (CN); Tiansheng HUANG, Zhuhai (CN); Yuting LIU, Zhuhai (CN); Zhenxing WANG, Zhuhai (CN); Songhong DING, Zhuhai (CN)

(21) Appl. No.: 18/207,163

(22) Filed: Jun. 8, 2023

(30) Foreign Application Priority Data
Jan. 20, 2023 (CN) 202320160061.9

(51) Int. Cl. A61L 2/14; A61L 9/22

(52) U.S. Cl. CPC A61L 2/14; A61L 9/22

(57) ABSTRACT

The disclosure provides a plasma module, a disinfection and sterilization apparatus, and an air duct assembly. An outer surface of the plasma module at least includes a domed surface and a flat surface, an edge of the domed surface being in sealed connection with an edge of the flat surface. The plasma module further includes a plasma emission probe, at least a part of the plasma emission probe being exposed outside an outer surface of the domed surface; and a plasma generator, the plasma generator being arranged inside the plasma module, and the plasma generator being electrically connected to a part, located inside the plasma module, of the plasma emission probe. By using the plasma module, the wind resistance is reduced, and the flow efficiency of positive and negative ions generated by the plasma generator is improved, so that the sterilization efficiency is increased.

### CROSS-REFERENCE TO RELATED APPLICATION

This disclosure claims priority to Patent Application No. 202320160061.9, filed to China National Intellectual Property Administration on Jan. 20, 2023 and entitled “Plasma Module, Disinfection and Sterilization Apparatus, and Air Duct Assembly”, the disclosure of which is hereby incorporated by reference in its entirety.

### TECHNICAL FIELD

The disclosure relates to the related technical field of plasma generating devices, in particular to a plasma module, a disinfection and sterilization apparatus, and an air duct assembly.

### BACKGROUND

An air ionizer achieves a disinfection and sterilization effect by emitting positive and negative ions into air, and generally includes an electrode that applies a high voltage. Gas molecules near the electrode are ionized when they obtain or lose electrons. Because the ions carry a charge of the nearest electrode, they are repelled from the electrode like charge repulsion. In a typical air ionizer, airflow is introduced into a device to carry the ions from the electrode to a “target area” where the ion content needs to be increased.

In order to achieve an effect of disinfecting and sterilizing the environment, the current mainstream way is to drive the flow of the positive and negative ions using gas flow, for example, some plasma generators are matched with a liquid air duct to facilitate the flow of the positive and negative ions, so as to drive the movement of the positive and negative ions through the gas flow inside the air duct. In this way, the positive and negative ions may not only disinfect and sterilize the air inside the air duct, but also follow the gas to flow into the ambient gas outside the air duct to disinfect and sterilize the ambient gas.

The air ionizer is applied to a variety of different air and fluids, and follows the air to flow. However, the flow rate of the positive and negative ions generated by the existing ion generator is greatly affected by an ion generator body, which affects the disinfection and sterilization efficiency.

It may be seen from the above that the current air ionizer has the problem of low disinfection and sterilization efficiency.

### SUMMARY

Some embodiments of the disclosure are to provide a plasma module, a disinfection and sterilization apparatus, and an air duct assembly, so as to solve the problem of low disinfection and sterilization efficiency of an air ionizer in the related art.

In order to achieve the above-mentioned object, according to one embodiment of the disclosure, a plasma module is provided. An outer surface of the plasma module at least includes a domed surface and a flat surface, an edge of the domed surface being in sealed connection with an edge of the flat surface. The plasma module further includes a plasma emission probe, at least a part of the plasma emission probe being exposed outside an outer surface of the domed surface; and a plasma generator, the plasma generator being arranged inside the plasma module, and the plasma generator being electrically connected to a part, located inside the plasma module, of the plasma emission probe.

In an embodiment mode, an edge of the domed surface is circular or elliptical or polygonal, and when an edge of the domed surface is polygonal, the number of sides of a polygon is greater than or equal to or 4.

In an embodiment mode, the domed surface is a smooth hemispherical surface or a smooth semi-ellipsoidal surface; or the domed surface is spliced by a plurality of polygons to form a hemispherical or semi-ellipsoidal surface.

In an embodiment mode, the plasma module further includes a housing. The housing is provided with the domed surface, an accommodating cavity is arranged inside the housing, and the plasma generator is inside the accommodating cavity.

In an embodiment mode, the plasma module further includes a colloid part. The colloid part is arranged inside the accommodating cavity and wraps the plasma generator, one side of the colloid part toward an opening of the housing is provided with the flat surface, and the flat surface is arranged approximately flush with the opening of the housing.

In an embodiment mode, the plasma module further includes a bottom plate. The bottom plate is arranged at the opening of the housing, and an outer periphery of the bottom plate is in sealed connection with an edge of the opening, so that the accommodating cavity becomes a sealed cavity. The bottom plate is provided with the flat surface. A glue injection hole in communication with the sealed cavity is reserved in the housing and/or the bottom plate.

In an embodiment mode, the domed surface is provided with a windward side and a leeward side in a direction of air flow, and the plasma emission probe is arranged on the leeward side; and/or the plasma emission probe obliquely protrudes in a direction toward the leeward side with respect to the flat surface.

In an embodiment mode, an angle α is formed between the flat surface and the plasma emission probe, a scope of the angle α meets 5° to 30°.

In an embodiment mode, the plasma emission probe includes a positive ion emission probe and a negative ion emission probe which are arranged in pairs and symmetrically, and a distance between the positive ion emission probe and the negative ion emission probe is not less than 50 mm.

In an embodiment mode, there is one or more groups of positive ion emission probes and negative ion emission probes arranged in pairs and symmetrically, and when there are a plurality of groups of positive ion emission probes and negative ion emission probes arranged in pairs and symmetrically, the positive ion emission probes and/or negative ion emission probes of different groups of the plurality of groups are spaced apart from each other.

In an embodiment mode, a distance between the positive ion emission probe and the negative ion emission probe in different groups of the plurality of groups is greater than 50 mm; and/or the distance between the positive ion emission probe and the negative ion emission probe in different groups of the plurality of groups is greater than a distance between the positive ion emission probe and the negative ion emission probe in the same group of the plurality of groups.

In an embodiment mode, the plasma module further includes a mounting flange. The mounting flange is integrally formed on the domed surface, one end surface of the mounting flange is coplanar with the flat surface, and the flat surface serves as a mounting surface.

In an embodiment mode, one or more mounting flanges are arranged, and when a plurality of mounting flanges are arranged, the plurality of mounting flanges are arranged at equal intervals in a circumferential direction of the flat surface.

In an embodiment mode, the mounting flange is provided with a mounting groove, and the plasma module further includes a magnet, the magnet being arranged inside the mounting groove.

In an embodiment mode, the mounting flange is provided with a mounting hole, the magnet is of an annular structure, the magnet is provided with a center hole coaxial with the mounting hole, a aperture of the center hole is not less than that of the mounting hole, and the center hole and the mounting hole are configured to enable a fastener to pass through.

In an embodiment mode, the mounting groove and the magnet are clamped or bonded.

In an embodiment mode, a mounting pipe is arranged on the domed surface, the mounting pipe is configured to mount the plasma emission probe, and one end of the plasma emission probe protrudes from the inside of the plasma module through the mounting pipe to the outside of the domed surface.

In an embodiment mode, the mounting pipe protrudes from the domed surface in a direction away from the domed surface, and an inner wall surface of one end, away from the domed surface, of the mounting pipe abuts against an outer cladding layer of the plasma emission probe.

In an embodiment mode, an inner diameter of the mounting pipe decreases gradually in a direction away from the domed surface, and a necking channel is formed in the mounting pipe.

In an embodiment mode, the mounting pipe is a telescopic pipe; and/or the mounting pipe is a flexible pipe to facilitate adjustment of an angle of the mounting pipe with respect to the domed surface; and/or an outer wall surface of the mounting pipe and the domed surface are integrally formed.

In an embodiment mode, the mounting pipe is angularly adjustably connected to the domed surface, the mounting pipe includes a joint section and a mounting section which are connected in sequence, the joint section and the mounting section together forming a channel configured to enable the plasma emission probe to pass through, and the joint section being pivotally connected to the domed surface; and/or the joint section is pivotally connected to the mounting section.

In an embodiment mode, the plasma module further includes an indicator lamp and a circuit board. The indicator lamp is mounted on the domed surface, and both the indicator lamp and the plasma generator are electrically connected to the circuit board, and the indicator lamp is configured to indicate an operating state of the plasma generator.

In an embodiment mode, a distance between the indicator lamp and the edge of the domed surface is equal to the distance between the plasma emission probe and the edge of the domed surface; or the distance between the indicator lamp and the edge of the domed surface is less than the distance between the plasma emission probe and the edge of the domed surface; or the distance between the indicator lamp and the edge of the domed surface is greater than the distance between the plasma emission probe and the edge of the domed surface.

In an embodiment mode, the plasma module further includes a wire. A terminal of the wire penetrates through the domed surface and extends into the plasma module to be electrically connected to the circuit board.

According to another embodiment of the disclosure, a disinfection and sterilization apparatus is provided. The disinfection and sterilization apparatus includes the above-mentioned plasma module.

According to another embodiment of the disclosure, an air duct assembly is provided. The air duct assembly includes the above-mentioned plasma module.

In an embodiment mode, the air duct assembly includes an air duct, the plasma module is arranged on the air duct, an angle β is formed between a direction of air flow of the air duct and a protruding direction of the plasma emission probe,
a scope of the angle βmeets: 95°≤β≤120°; or a scope of the angle βmeets: 60°≤β≤85°.

In an embodiment mode, the scope of the angle βmeets: 95°≤β≤100; or the scope of the angle βmeets: 105°≤β≤120°; or the scope of the angle βmeets: 60≤β≤75″; or the scope of the angle βmeets: 80°≤β≤85°.

By applying the technical solution of the disclosure, the outer surface of the plasma module at least includes the domed surface and the flat surface, the edge of the domed surface being in sealed connection with the edge of the flat surface. The plasma module further includes the plasma emission probe and the plasma generator. At least a part of the plasma emission probe is exposed outside the outer surface of the domed surface. The plasma generator is arranged inside the plasma module, and the plasma generator is electrically connected to a part, located inside the plasma module, of the plasma emission probe.

The outer surface of the plasma module of the disclosure adopts a structure in which the domed surface is matched with the flat surface, so as to achieve that, during use, when wind blows to the domed surface from any direction, compared with the related art, the domed surface of the disclosure has an effect of reducing the wind resistance, so that no limitation is made to the mounting of the plasma emission probe in the disclosure. The plasma emission probe is able to be mounted at any position in the circumferential direction of the domed surface, and the problem that the wind resistance affects the flow of the positive and negative ions does not occur. The flow efficiency of air is provided by reducing the wind resistance, so that the flow speed of the positive and negative ions discharged from the plasma emission probe is increased to achieve the efficiency of the positive and negative ions entering the ambient gas, thereby effectively improving the disinfection and sterilization effect and enhancing the user experience.

It is to be noted that, when the plasma module of the disclosure is actually mounted inside an air duct, the flowing gas in the air duct drives the flow of the positive and negative ions generated by the plasma emission probe. The positive and negative ions located inside the air duct also have a combined phenomenon during the flow process, the combined positive and negative ions are discharged to disinfect and sterilize the inside of the air duct, and the positive and negative ions following the gas into the ambient air achieve the disinfection and sterilization of the air in the external environment, so that the plasma module of the disclosure not only achieve the disinfection and sterilization of the ambient air, but also achieve the disinfection and sterilization of the gas inside the air duct, thereby improving the sterilization efficiency.

### BRIEF DESCRIPTION OF THE DRAWINGS

The accompanying drawings constituting a part of the disclosure are used to provide a further understanding of the disclosure, and the exemplary embodiments of the disclosure and the description thereof are used to explain the disclosure, but do not constitute improper limitations to the disclosure. In the accompanying drawings:

FIG. 1 shows a three-dimensional structure diagram of a plasma module according to a first embodiment of the disclosure.

FIG. 2 shows another three-dimensional structure diagram of a plasma module according to a first embodiment of the disclosure.

FIG. 3 shows a three-dimensional structure diagram of a plasma module according to a second embodiment of the disclosure.

FIG. 4 shows another three-dimensional structure diagram of a plasma module according to a second embodiment of the disclosure.

FIG. 5 shows a mounting structure diagram of a plasma emission probe according to a second embodiment and a third embodiment of the disclosure.

FIG. 6 shows a front view of a plasma module according to a second embodiment and a third embodiment of the disclosure.

FIG. 7 shows a structural diagram of one implementation mode of a plasma emission probe according to a third embodiment of the disclosure.

FIG. 8 shows a structural diagram of another implementation mode of a plasma emission probe according to a third embodiment of the disclosure.

FIG. 9 shows a mounting relationship diagram of a plasma emission probe of FIG. 8.

FIG. 10 shows a front view of a plasma module according to a first embodiment of the disclosure, a mounting pipe being a telescopic pipe.

FIG. 11 shows a top view of a plasma module according to a second embodiment of the disclosure.

FIG. 12 shows a top view of a plasma module according to a fourth embodiment of the disclosure.

FIG. 13 shows a front view of a plasma module according to a fifth embodiment of the disclosure.

FIG. 14 shows a three-dimensional structure diagram of a plasma module according to a fifth embodiment of the disclosure.

FIG. 15 shows a three-dimensional structure diagram of a plasma module according to an eighth embodiment of the disclosure.

FIG. 16 shows another three-dimensional structure diagram of a plasma module according to an eighth embodiment of the disclosure.

FIG. 17 shows another three-dimensional structure diagram of a plasma module according to an eighth embodiment of the disclosure.

FIG. 18 shows a position relationship diagram of a plasma emission probe of a plasma module and a fan of the disclosure.

FIG. 19 shows a position relationship diagram of a plasma emission probe of a plasma module and a fan of the disclosure, wherein α=0 degree.

FIG. 20 shows a position relationship diagram of a plasma emission probe of a plasma module and a fan of the disclosure, wherein α=90 degree.

FIG. 21 shows a diagram of a comparison test of an ion concentration of a plasma emission placed at different angles.

FIG. 22 shows a position relationship diagram of a plasma emission probe of a plasma module and a fan in another embodiment of the disclosure.

Herein, the above-mentioned accompanying drawings include the following reference signs:
10. Housing; 110. Domed surface; 20. Colloid part; 210. Flat surface; 30. Plasma emission probe; 310. Positive ion emission probe; 320. Negative ion emission probe; 40. Mounting flange; 410. Mounting groove; 420. Mounting hole; 50. Mounting pipe; 510. Joint section; 520. Mounting section; 60. Indicator lamp; 70. Magnet; 710. Center hole; 80. Wire; 90. Plasma generator; and 100. Circuit board; 1000. Fan; 1001. Air inlet of fan.

### DETAILED DESCRIPTION OF THE EMBODIMENTS

It is to be noted that embodiments in the disclosure and features in the embodiments may be combined under the condition of no conflicts. The disclosure is described below with reference to the drawings and in conjunction with the embodiments in detail.

It is to be noted that, unless otherwise specified, all technical and scientific terms used herein have the same meaning as commonly understood by those of ordinary skill in the art of the disclosure.

In the disclosure, the orientation words used, such as “up, down, top and bottom”, are generally for the directions shown in the accompanying drawings or for vertical, perpendicular or gravity directions, without being otherwise stated; and likewise, for ease of understanding and description, “inside and outside” refer to the inside and outside of the contour of the components themselves, but the above-mentioned orientation words are not intended to limit the disclosure.

### Embodiment 1

In order to solve the problem of low disinfection and sterilization efficiency of an air ionizer in the related art, the disclosure provides a plasma module. The plasma module is able to be applied to the household or industrial field.

As shown in FIGS. 1, 2 and 10, an outer surface of the plasma module at least includes a domed surface 110 and a flat surface 210, an edge of the domed surface 110 being in sealed connection with an edge of the flat surface 210. The plasma module further includes a plasma emission probe 30 and a plasma generator 90. At least a part of the plasma emission probe 30 is exposed outside an outer surface of the domed surface 110. The plasma generator 90 is arranged inside the plasma module, and the plasma generator 90 is electrically connected to a part, located inside the plasma module, of the plasma emission probe 30.

Specifically, an outer surface of the plasma module of the disclosure adopts a structure in which the domed surface 110 is matched with the flat surface 210, so as to achieve that, during use, when wind blows to the domed surface 110 from any direction, compared with the related art, the domed surface 110 of the disclosure has an effect of reducing the wind resistance, so that no limitation is made to the mounting of the plasma emission probe 30 in the disclosure. The plasma emission probe 30 is able to be mounted at any position in the circumferential direction of the domed surface 110, and the problem that the wind resistance affects the flow of the positive and negative ions does not occur. The flow efficiency of air is provided by reducing the wind resistance, so that the flow speed of the positive and negative ions discharged from the plasma emission probe 30 is increased to achieve the efficiency of the positive and negative ions entering the ambient gas, thereby effectively improving the disinfection and sterilization effect and enhancing the user experience.

The plasma module of the disclosure is applicable to be mounted in different air duct scenes, and the plasma module of the disclosure has the characteristic of wide invention range.

It is to be noted that, when the plasma module of the disclosure is actually mounted inside an air duct, the flowing gas in the air duct drives the flow of the positive and negative ions generated by the plasma emission probe 30. The positive and negative ions located inside the air duct also have a combined phenomenon during the flow process, the combined positive and negative ions are discharged to disinfect and sterilize the inside of the air duct, and the positive and negative ions following the gas into the ambient air achieve the disinfection and sterilization of the air in the external environment, so that the plasma module of the disclosure not only achieve the disinfection and sterilization of the ambient air, but also achieve the disinfection and sterilization of the gas inside the air duct, thereby improving the sterilization efficiency.

Further, the plasma generator 90 emits the positive and negative ions to the outside of the domed surface 110 through the plasma emission probe 30, and the positive and negative ions enter the air to achieve the technical effect of plasma sterilization.

In this embodiment, an edge of the domed surface 110 is in sealed connection with an edge of the flat surface 210, so that the domed surface 110 and the flat surface 210 are seamlessly connected and matched to form a whole body, thereby preventing liquid and gas from entering the inside of the plasma module.

It is to be noted that, in a general design, the plasma generator 90 that emits a greater number of the negative ions than the positive ions is preferably selected, and the concentration of the negative ions emitted from the plasma generator 90 is controlled to be greater than the concentration of the positive ions, so as to ensure a high concentration of the negative ions in the air, so that the negative ions exists in the air for a long time to purify and improve the quality of the air.

A carbon fiber brush probe is used for the plasma emission probe 30, and since the carbon fiber brush probe has a large contact area with the air, more plasma is generated, which greatly increases the sterilization efficiency.

Of course, the plasma emission probe 30 is a needle-like or tubular electrode. The plasma emission probe 30 is able to be of other structures by just ensuring that the plasma emission probe 30 is able to emit the positive or negative ions.

It should be further explained that the instant that the positive and negative ions generated by the plasma generator 90 are neutralized in the air generates the release of energy, resulting in changes in the structure of the surrounding bacteria or the conversion of energy, so that the bacteria die to achieve the bactericidal effect.

As shown in FIGS. 1, 2 and 10, an edge of the domed surface 110 in the disclosure is circular or elliptical or polygonal, and when an edge of the domed surface 110 is polygonal, the number of sides of a polygon is greater than or equal to or 4.

The domed surface 110 in the embodiment is a hemispherical surface. The domed surface 110 is able to be a smooth hemispherical surface, and the domed surface 110 is able to also be spliced by a plurality of polygons to form a hemispherical surface, so that the whole domed surface 110 is hemispherical.

In the embodiment, the plasma module further includes a housing 10. The housing 10 provides the domed surface 110, an accommodating cavity is arranged inside the housing 10, and the plasma generator 90 is inside the accommodating cavity. The present embodiment provides the following two implementation modes according to the different structures that provide the flat surface 210.

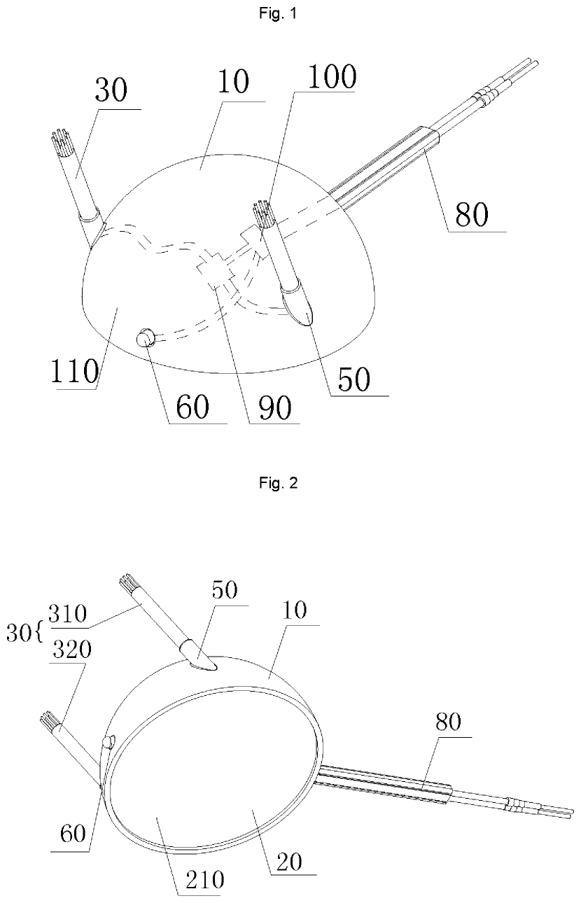

In the specific implementation mode shown in FIG. 2, the plasma module further includes a colloid part 20. The colloid part 20 is arranged inside the accommodating cavity and wraps the plasma generator 90, one side of the colloid part 20 toward an opening of the housing 10 provides the flat surface 210, the flat surface 210 is arranged approximately flush with the opening of the housing 10, and the flat surface 210 serves a mounting surface.

It is to be noted that, the colloid part 20 of the disclosure fills the accommodating cavity by means of glue injection. The colloid part 20 is able to be epoxy resin, so as to wrap and fix the plasma generator 90, thereby achieving the effect of positioning and protection. During the actual glue filling process, in order to prevent the colloid part 20 from overflowing the accommodating cavity, the flat surface 210 provided by the colloid part 20 is able to be slightly lower than an edge of the domed surface 110 so as to be approximately flush.

In a non-illustrated specific implementation mode of the embodiment, the plasma module further includes a bottom plate. The bottom plate is arranged at the opening of the housing 10, and the outer periphery of the bottom plate is in sealed connection with an edge of the opening, so that the accommodating cavity becomes a sealed cavity. The bottom plate provides the flat surface 210. A glue injection hole in communication with the sealed cavity is reserved in the housing 10 and/or the bottom plate.

The sealed cavity is enclosed by the bottom plate and the housing 10, and then the glue is injected into the sealed cavity, so as to fill the inside of the sealing cavity with glue, and the glue is able to be epoxy resin, so as to wrap the plasma generator 90, thereby achieving the effect of positioning protection.

As shown in FIGS. 1, 2 and 10, the positive and negative ions generated by the plasma module of the disclosure follow the air to flow, and therefore the domed surface 110 is provided with a windward side and a leeward side in a direction of air flow.

Specifically, in order to prevent the domed surface 110 from interfering with the flow of the positive and negative ions, the plasma emission probe 30 is arranged on the leeward side. The gas flows from the windward side toward the leeward side, and carries the positive and negative ions emitted by the plasma emission probe 30 on the leeward side into the air.

Further, the plasma emission probe 30 is arranged on the leeward side, so as to prevent the positive and negative ions from being affected by the domed surface 110 during the process of flowing with the gas.

In the embodiment, the plasma emission probe 30 obliquely protrudes in a direction toward the leeward side with respect to the flat surface 210.

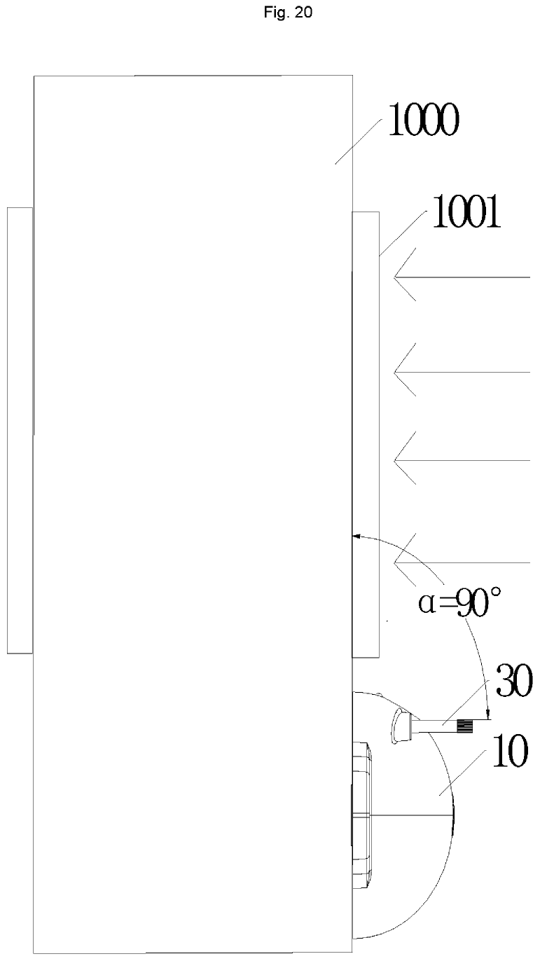

In the disclosure, the air duct assembly includes air duct, the plasma module is arranged on the air duct, an angle β is formed between a direction of air flow of the air duct and a protruding direction of the plasma emission probe. As shown in FIGS. 18 and 20, a position relationship diagram of the plasma emission probe 30 of the plasma module and a fan 1000 of the disclosure. the air duct assembly includes the fan 1000, the fan has the air duct. Moreover, an angle α is formed between the flat surface 210 and the plasma emission probe 30. At the same time, the angle α is also formed between the plasma emission probe 30 and a plane of the air inlet 1001. In the present embodiment, an air inlet 1001 is formed in the fan 1000, the plasma module is located at the air inlet 1001.

The disclosure is provided with four schemes (option A to option D) to test. The disclosure is provided with Table 1. Wherein, the test conditions: the temperature is 23.7° C.; the humidity is 37% RH; the wind speed is 6 m/s; the test distance is 2 meters away from the air outlet of the fan.

Table 1 is the comparison test of the ion concentration of the plasma emission probe placed at different angles.

| | Technical scheme | | | | | | | |
|---|---|---|---|---|---|---|---|---|
| | Option A | | Option B | | Option C | | Option D | |
| Angle α | 5° to 10° | | 15° to 30° | | 45° to 60° | | 90° | |
| Angle β | 95° to 100° | | 105° to 120° | | 135° to 150° | | 180° | |
| FIG. | 18 | | 18 | | | | 20 | |
| concentration | Positive | Negative | Positive | Negative | Positive | Negative | Positive | Negative |
| (10⁶/cm³) | ions | ions | ions | ions | ions | ions | ions | ions |
| | 1.9 | 2.5 | 1.8 | 2.5 | 1.7 | 2.2 | 1.6 | 2.8 |

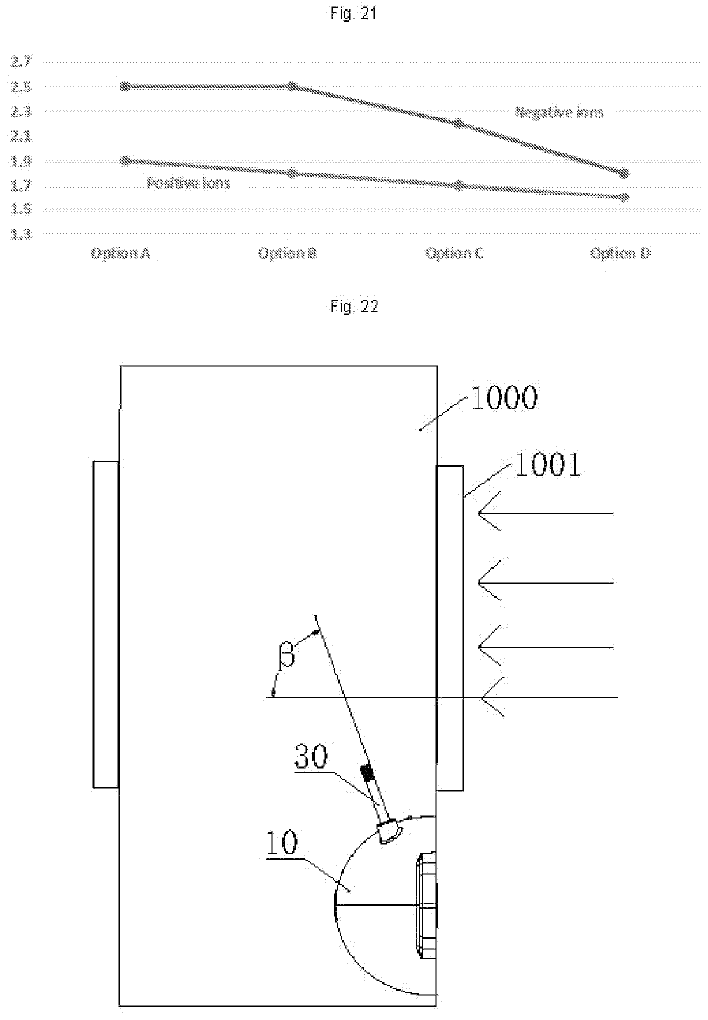

FIG. 21 shows the result of the comparison test of the ion concentration of the plasma emission placed at different angles. From FIG. 21 we can know that the angle & is smaller and the ion concentration is higher. Based on the tests of the above four schemes, it is recommended to choose “Option A” (the scope of the angle α is 5° to 10°) and “Option B” (the scope of the angle α is 15° to 30°).

That is to say, the scope of the angle α is better 5° to 30°, and the scope of the angle β meets: 95°≤β≤120°. Further, the scope of the angle β meets: 95°≤β≤100° or 105°≤β≤120°.

The disclosure is further provided with four schemes (option E to option H) to test. The disclosure is provided with Table 2. Wherein, the test conditions: the temperature is 23.7° C.; the humidity is 37% RH; the wind speed is 6 m/s; the test distance is 2 meters away from the air outlet of the fan. In FIG. 22, in another embodiment, a mounting position of the plasma module on the air duct is changed relative to the embodiment shown on FIG. 18. In FIG. 22, the plasma emission probe is extending to the upper left, the scope of the angle β meets: 60°≤β≤85°.

Table 2 is the comparison test of the ion concentration of the plasma emission probe placed at different angles.

| | Technical scheme | | | | | | | |
|---|---|---|---|---|---|---|---|---|
| | Option E | | Option F | | Option G | | Option H | |
| Angle β | 80° to 85° | | 60° to 75° | | 30° to 45° | | 0° | |
| FIG. | | | | | 22 | | | |
| concentration | Positive | Negative | Positive | Negative | Positive | Negative | Positive | Negative |
| (10⁶/cm³) | ions | ions | ions | ions | ions | ions | ions | ions |
| | 1.9 | 2.5 | 1.8 | 2.5 | 1.7 | 2.2 | 1.6 | 2.8 |

From Table 2 we can know that the angle β is bigger and the ion concentration is higher. Based on the tests of the above four schemes, it is recommended to choose “Option E” (the scope of the angle β is 80° to 85°) and “Option F” (the scope of the angle β is 60° to 75°).

That is to say, the scope of the angle (meets: 80° to 85°. After testing, by changing the mounting position of the plasma module to make (satisfy 60° to 75°, the same beneficial effect as that of the above embodiment may be obtained. That is to say, the range of the angle β between the direction of air flow of the air duct and the protruding direction of the plasma emission probe is a key point affecting beneficial effects.

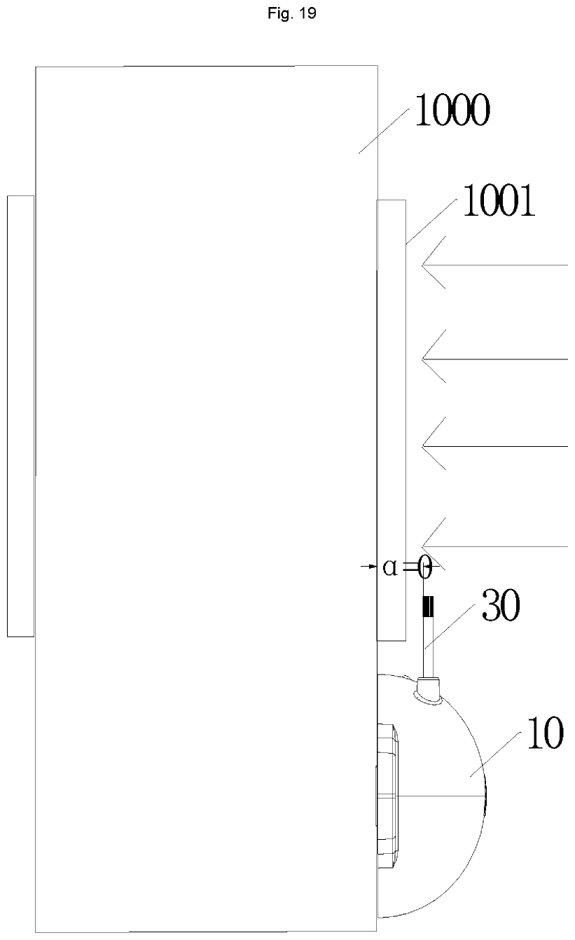

It should be noted that FIG. 19 is another embodiment, in FIG. 19, the angle α of the plasma emission probe 30 and the air inlet 1001 is 0 degree, the plasma concentration is the highest, but the arrangement position of the plasma emission probe 30 and the air inlet 1001 has the installation interference problem. So, the plasma emission probe 30 obliquely protrudes in the direction toward the leeward side with respect to the flat surface 210.

In FIG. 19, α=0 degree, the air flow direction is perpendicular to the protruding direction of the plasma emission probe 30.

In FIG. 20, α=90 degree, the air flow direction opposites the protruding direction of the plasma emission probe 30.

It should be noted that, in the above embodiments, α and β satisfy the relationship of β=90°+α or β=90°−α. However, in other embodiments, when the mounting relationship of the plasma module in the air duct is changed, the angle relationship between α and β is changed. Therefore, the angle relationship between α and β should be based on actual mounting situations, and it does not mean that α and β must be satisfy β=90°+α or β=90°−α.

As shown in FIGS. 1, 2 and 10, the plasma emission probe 30 includes a positive ion emission probe 310 and a negative ion emission probe 320 arranged in pairs and symmetrically, and a distance between the positive ion emission probe 310 and the negative ion emission probe 320 is not less than 50 mm.

The positive ion emission probe 310 emits the positive ions, and the negative ion emission probe 320 emits the negative ions. In order to prevent the generation of ozone due to the ionization phenomenon of the positive and negative ions crossing in space, the distance between the positive ion emission probe 310 and the negative ion emission probe 320 is not less than 50 mm.

Specifically, there is one or more groups of positive ion emission probes 310 and negative ion emission probes 320 arranged in pairs and symmetrically, and when there are a plurality of groups of positive ion emission probes 310 and negative ion emission probes 320 arranged in pairs and symmetrically, the positive ion emission probes 310 and/or negative ion emission probes 320 of different groups are spaced apart from each other.

In the embodiment, the distance between the positive ion emission probe 310 and the negative ion emission probe 320 in different groups of the plurality of groups is greater than 50 mm.

In the embodiment, the distance between the positive ion emission probe 310 and the negative ion emission probe 320 in different groups of the plurality of groups is greater than the distance between the positive ion emission probe 310 and the negative ion emission probe 320 in the same group of the plurality of groups.

As shown in FIGS. 1, 2 and 10, the domed surface 110 is provided with a mounting pipe 50, the mounting pipe 50 is configured to mount the plasma emission probe 30, and one end of the plasma emission probe 30 protrudes from the inside of the plasma module through the mounting pipe 50 to the outside of the domed surface 110.

The mounting pipe 50 protrudes from the domed surface 110 in a direction away from the domed surface 110, and an inner wall surface of one end, away from the domed surface 110, of the mounting pipe 50 abuts against an outer cladding layer of the plasma emission probe 30.

Specifically, the inner diameter of the mounting pipe 50 decreases gradually in a direction away from the domed surface 110, a necking channel is formed in the mounting pipe 50, and the plasma emission probe 30 is mounted after passing through the necking channel.

In the embodiment, an outer wall surface of the mounting pipe 50 and the domed surface 110 are integrally formed.

Specifically, in order to achieve length adjustment, when the outer wall surface of the mounting pipe 50 and the domed surface 110 are integrally formed, the mounting pipe 50 as shown in FIG. 10 is a telescopic pipe.

Of course, no limitation is made to the structure in which the mounting pipe 50 is the telescopic pipe, the mounting pipe 50 is able to be a flexible pipe. An angle of the mounting pipe 50 with respect to the domed surface 110 is adjusted by bending the flexible pipe.

As shown in FIGS. 1 and 2, the plasma module further includes an indicator lamp 60 and a circuit board 100. The indicator lamp 60 is mounted on the domed surface 110, and both the indicator lamp 60 and the plasma generator 90 are electrically connected to the circuit board 100, and the indicator lamp 60 is configured to indicate an operating state of the plasma generator 90.

Specifically, the distance between the indicator lamp 60 and the edge of the domed surface 110 is less than the distance between the plasma emission probe 30 and the edge of the domed surface 110.

Further, the indicator lamp 60 has different display colors, and the circuit board 100 controls the indicator lamp 60 to display different colors so as to feed back the operating state of the plasma generator 90, for example, when the indicator lamp 60 displays red, the plasma generator 90 is in a shutdown state, and at this time, the plasma generator 90 does not generate the positive and negative ions; and when the indicator lamp 60 shows green, the plasma generator 90 is in the operating state, and the plasma module has a function of disinfection and sterilization.

It is to be noted that, the disclosure is provided with an alarm device, and the alarm device is in signal connection with the circuit board 100, so as to realize an alarm prompt through an alarm when the plasma generator 90 is switched from the operating state to the shutdown state.

In the embodiment, the plasma module further includes a wire 80. A terminal of the wire 80 penetrates through the domed surface 110 and extends into the plasma module to be electrically connected to the circuit board 100. The wire 80 is configured to provide a power supply.

### Embodiment 2

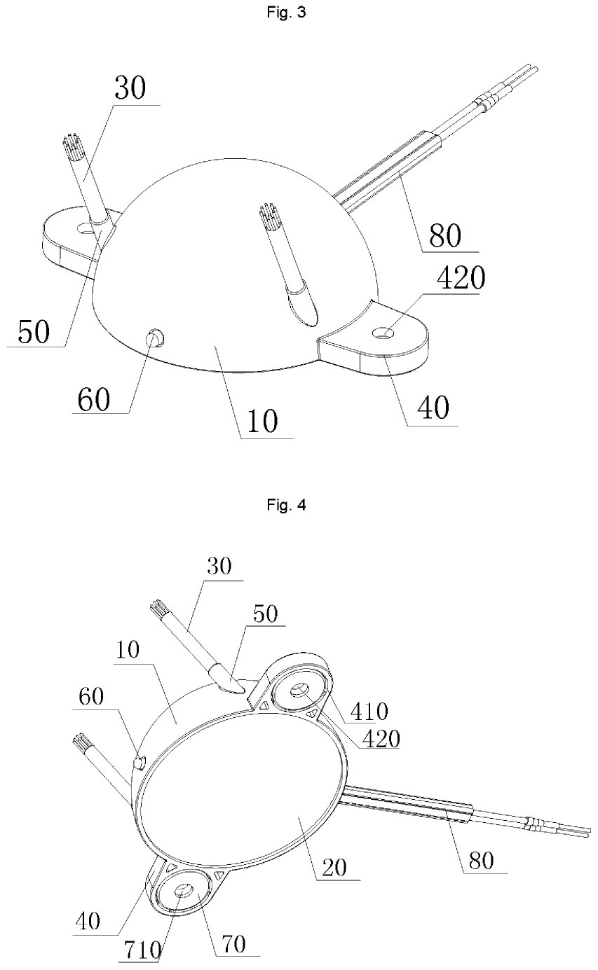

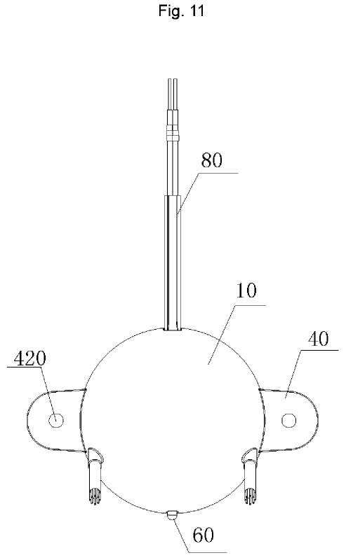

Different from Embodiment 1, in the embodiment, as shown in FIG. 3 to FIG. 11, the plasma module further includes a mounting flange 40. The mounting flange 40 is integrally formed on the domed surface 110, and one end surface of the mounting flange 40 is coplanar with the flat surface 210.

Specifically, one or more mounting flanges 40 are arranged, and when a plurality of mounting flanges 40 are arranged, the plurality of mounting flanges 40 are arranged at equal intervals in a circumferential direction of the flat surface 210.

Further, two, three, four, etc. mounting flanges 40 is arranged, and the mounting flanges 40 arranged at equal intervals have the technical effects of stable mounting and uniform stress.

In the embodiment, a bottom surface of the mounting flange 40 is flush with the flat surface, and a flange hole of the mounting flange 40 extends in a direction perpendicular to the flat surface 210.

As shown in FIG. 4, the mounting flange 40 is provided with a mounting groove 410, and the plasma module further includes a magnet 70, the magnet 70 being arranged inside the mounting groove 410.

The mounting flange 40 is provided with a mounting hole 420, the magnet 70 is of an annular structure, the magnet 70 is provided with a center hole 710 coaxial with the mounting hole 420, and the aperture of the center hole 710 is not less than that of the mounting hole 420. The plasma module is positioned by the mounting flange 40 specifically through a fastener. The fastener is one of a screw, a pin, etc. The fastener is mounted by penetrating through the mounting hole 420 and the center hole 710. The center hole 710 and the mounting hole 420 are configured to enable the fastener to pass through.

It is to be noted that, the magnet 70 has a function of positioning the fastener by means of a magnetic attraction fit with the fastener. Of course, in a case where the suction force of the magnet 70 is sufficiently large, the mounting flange 40 is mounted by means of the magnet 70 in magnetic attraction fit with a position to be mounted.

In the embodiment, the mounting groove 410 and the magnet 70 are clamped or bonded.

When the mounting groove 410 and the magnet 70 are clamped, the magnet 70 is mounted inside the mounting groove 410 by means of a conventional buckle. When the mounting groove 410 and the magnet 70 are mounted by means of bonding, glue is injected into the mounting groove 410, and then the magnet 70 is mounted inside the mounting groove 410 and completely bonded, or the magnet 70 is placed inside the mounting groove 410, and then glue is injected into the mounting groove 410 to achieve bonding.

### Embodiment 3

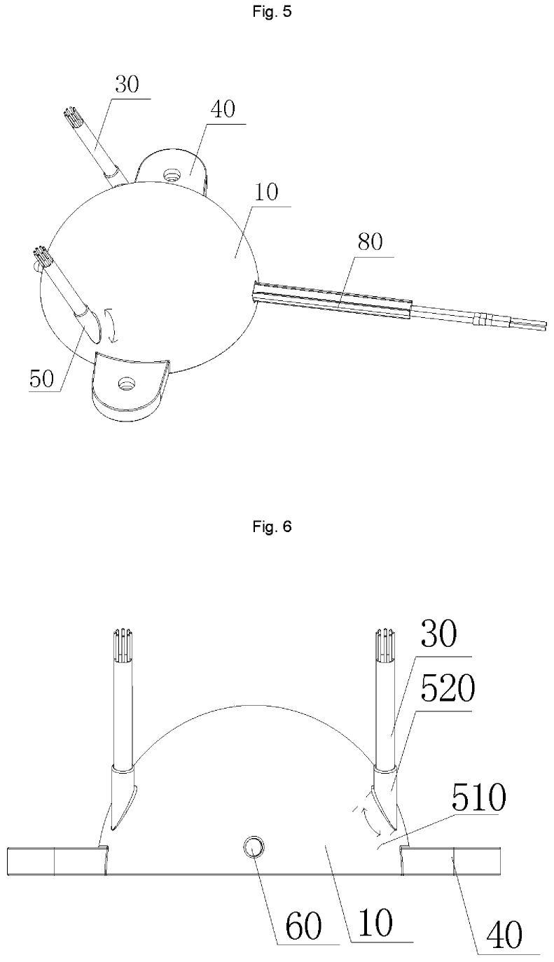

Different from Embodiment 1, in the embodiment, as shown in FIGS. 5 to 9, the mounting pipe 50 is angularly adjustably connected to the domed surface 110.

Specifically, the mounting pipe 50 includes a joint section 510 and a mounting section 520 which are connected in sequence, the joint section 510 and the mounting section 520 together forming a channel configured to enable the plasma emission probe 30 to pass through.

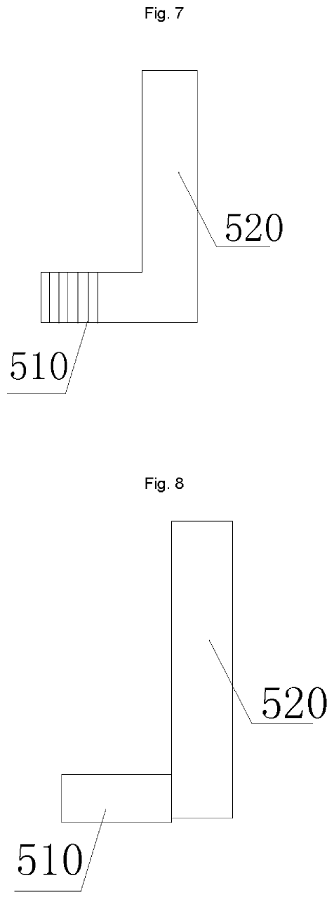

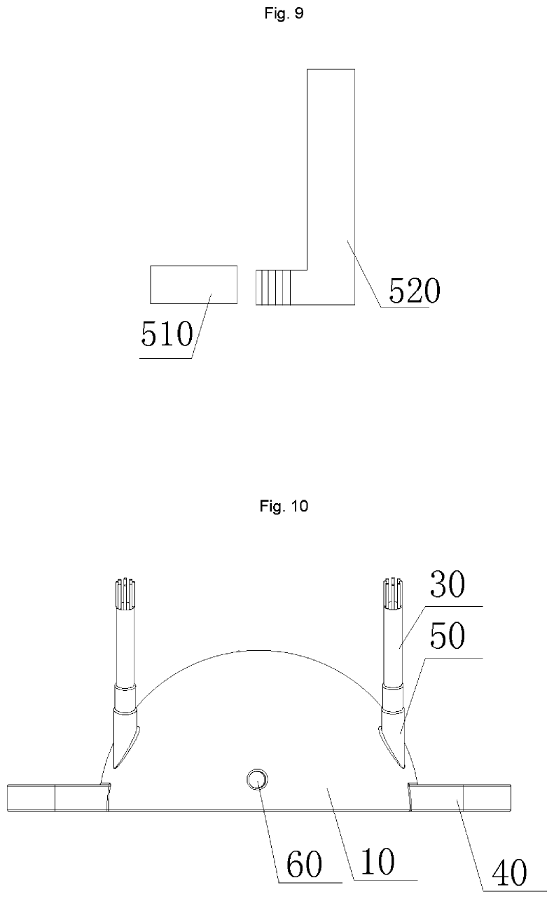

In the embodiment, an angle adjustment manner is that the joint section 510 is pivotally connected to the domed surface 110 as shown in FIG. 7, where the angle adjustment is achieved using a threaded structure, and is able to also be that the joint section 510 is pivotally connected to the mounting section 520 as shown in FIGS. 8 and 9, where the angle adjustment is achieved using the threaded structure.

### Embodiment 4

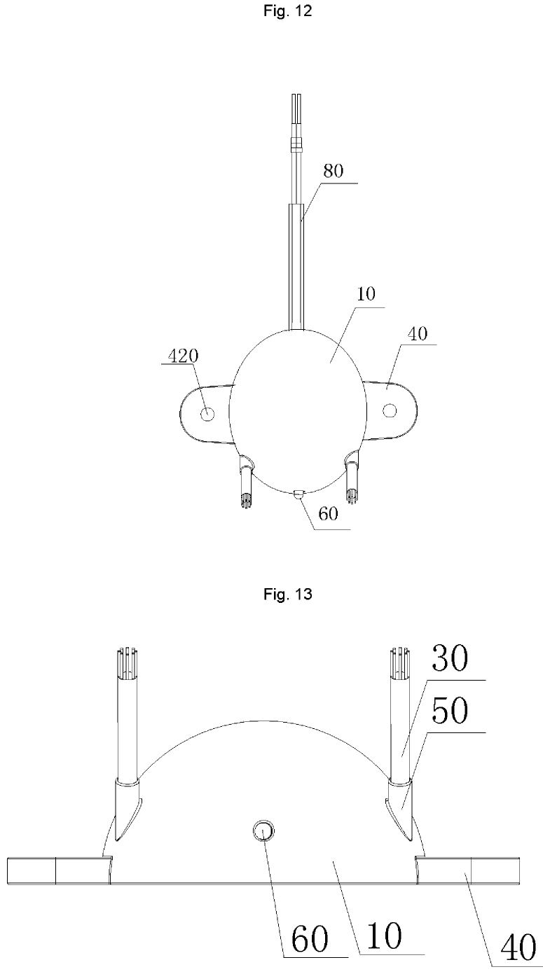

Different from Embodiment 1, in the embodiment, as shown in FIG. 12, the domed surface 110 is a semi-ellipsoidal surface, where the domed surface 110 is a smooth semi-ellipsoidal surface, or the domed surface 110 is able to be formed by splicing a plurality of polygons to form a semi-ellipsoidal surface.

When the domed surface 110 is semi-ellipsoidal, the edge of the domed surface 110 is elliptical.

### Embodiment 5

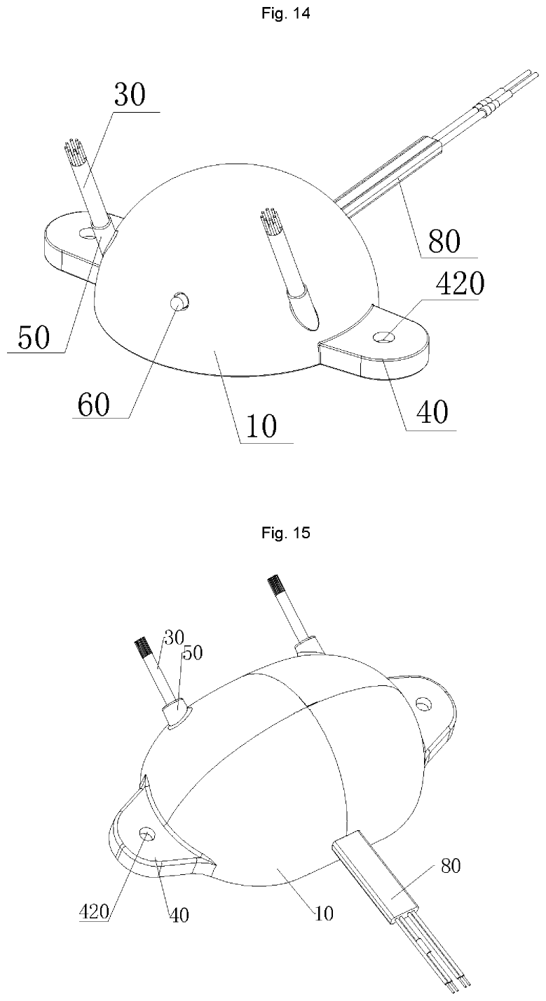

Different from Embodiment 1, in the embodiment, as shown in FIGS. 13 and 14, the distance between the indicator lamp 60 and the edge of the domed surface 110 is equal to the distance between the plasma emission probe 30 and the edge of the domed surface 110.

It is to be noted that, the distance between the indicator lamp 60 and the edge of the domed surface 110 is not limited to being equal to the distance between the plasma emission probe 30 and the edge of the domed surface 110, and in practical use, the distance between the indicator lamp 60 and the edge of the domed surface 110 may be greater than the distance between the plasma emission probe 30 and the edge of the domed surface 110.

### Embodiment 6

The embodiment provides a disinfection and sterilization apparatus. The disinfection and sterilization apparatus includes the plasma module of any one of Embodiments 1-5.

### Embodiment 7

The embodiment provides an air duct assembly. The air duct assembly includes the plasma module of any one of Embodiments 1-5.

The plasma module is mounted inside an air duct body of the air duct assembly, and positive and negative ions generated by the plasma module flow with airflow inside the air duct body.

In the embodiment, the air duct assembly is applied to a variety of devices in the household and industrial fields, for example, it is applied to an air conditioner, a refrigerator, a fan, etc.

### Embodiment 8

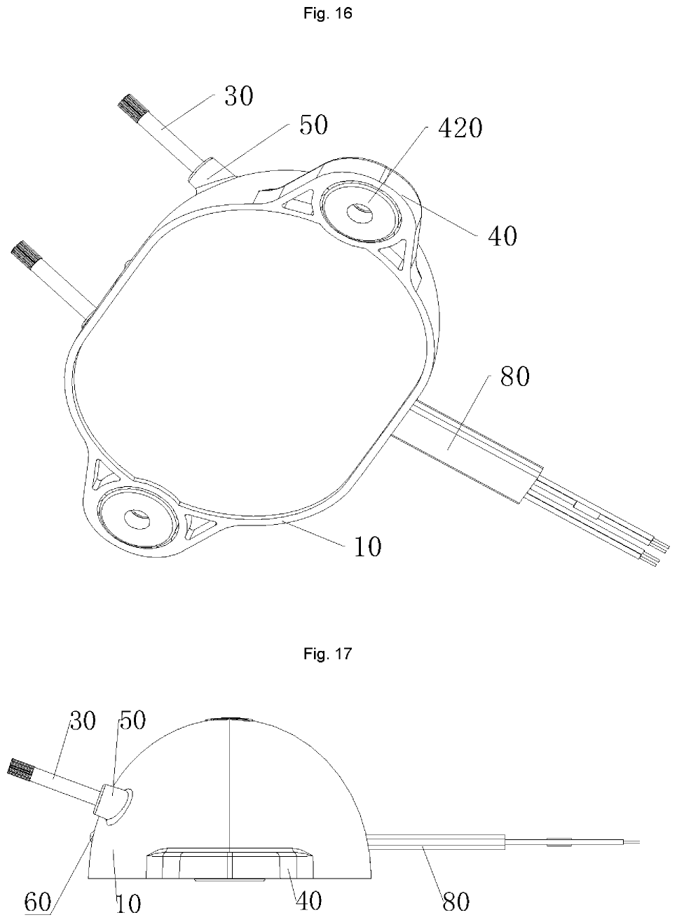

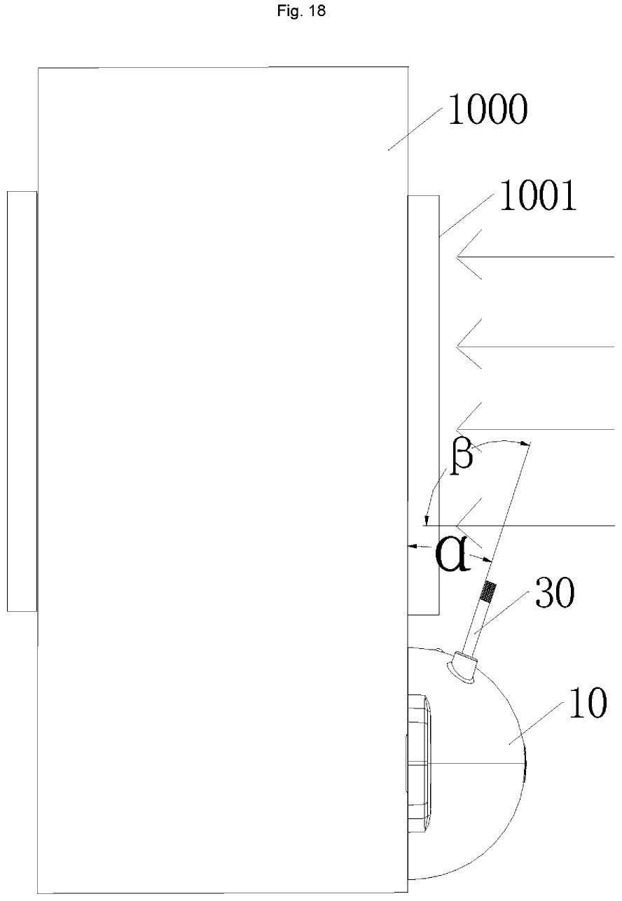

Different from Embodiment 1, in the embodiment, as shown in FIGS. 15 to 18, the domed surface 110 is not a hemispherical surface. The shape of the domed surface 110 is different from Embodiments 1 and 4. Moreover, the angle α=20 degree, the angle β=110 degree.

From the above description, it is able to be seen that the above-mentioned embodiments of the disclosure achieve the following technical effects.

The outer surface of the plasma module of the disclosure adopts a structure in which the domed surface 110 is matched with the flat surface 210, so as to achieve that, during use, when wind blows to the domed surface 110 from any direction, compared with the related art, the domed surface 110 of the disclosure has an effect of reducing wind resistance. The flow efficiency of air is provided by reducing the wind resistance, so that the flow speed of the positive and negative ions discharged from the plasma emission probe 30 is increased to achieve the efficiency of the positive and negative ions entering the ambient gas, thereby effectively improving the disinfection and sterilization effect and enhancing the user experience.

It is apparent that the described embodiments are only a part of the embodiments of the disclosure, and not all of them. All other embodiments obtained by those of ordinary skill in the art based on the embodiments of the disclosure without creative efforts are within the scope of the disclosure.

It is to be noted that the terms used herein are only for the purpose of describing the specific implementation modes and are not intended to limit the exemplary implementation modes of the disclosure. As used herein, singular forms are also intended to include plural forms as well, unless the context explicitly otherwise. It is also to be understood that, when used herein, the term “include” and/or “contain” indicates the existence of the features, steps, operations, devices, components and/or combinations of them.

It is to be noted that the terms “first”, “second” and the like in the description and claims of the disclosure and the above accompanying drawings are used to distinguish similar objects, and are not necessarily used to describe a specific order or sequence. It is to be understood that the data used in this way are interchangeable under appropriate circumstances, so that the implementation modes of the disclosure described herein is able to be implemented in an order other than those illustrated or described herein.

The above are only the preferred embodiments of the disclosure, and is not intended to limit the disclosure, and for those of ordinary skill in the art, various modifications and changes can be made to the disclosure. Any modifications, equivalent substitutions, improvements, etc. within the spirit and scope of the disclosure shall be included in the scope of protection of the disclosure.

What is claimed is:

1. A plasma module, wherein an outer surface of the plasma module at least comprises a domed surface and a flat surface, an edge of the domed surface being in sealed connection with an edge of the flat surface; the plasma module further comprises:
a plasma emission probe, at least a part of the plasma emission probe being exposed outside an outer surface of the domed surface; and
a plasma generator, the plasma generator being arranged inside the plasma module, and the plasma generator being electrically connected to a part, located inside the plasma module, of the plasma emission probe.

2. The plasma module according to claim 1, wherein the edge of the domed surface is circular or elliptical or polygonal, and when the edge of the domed surface is polygonal, the number of sides of a polygon is greater than or equal to or 4.

3. The plasma module according to claim 1, wherein,
the domed surface is a smooth hemispherical surface or a smooth semi-ellipsoidal surface; or
the domed surface is spliced by a plurality of polygons to form a hemispherical or semi-ellipsoidal surface.

4. The plasma module according to claim 1, further comprising a housing, wherein the housing is provided with the domed surface, an accommodating cavity is arranged inside the housing, and the plasma generator is inside the accommodating cavity.

5. The plasma module according to claim 4, further comprising a colloid part, wherein the colloid part is arranged inside the accommodating cavity and wraps the plasma generator, one side of the colloid part toward an opening of the housing is provided with the flat surface, and the flat surface is arranged approximately flush with the opening of the housing.

6. The plasma module according to claim 4, the plasma module further comprises a bottom plate, the bottom plate is arranged at the opening of the housing, and an outer periphery of the bottom plate is in sealed connection with an edge of the opening, so that the accommodating cavity becomes a sealed cavity, the bottom plate is provided with the flat surface, the glue injection hole in communication with the sealed cavity is reserved in the housing and/or the bottom plate.

7. The plasma module according to claim 4, wherein an angle α is formed between the flat surface and the plasma emission probe,
a scope of the angle α meets 5° to 30°.

8. The plasma module according to claim 1, wherein the plasma emission probe comprises a positive ion emission probe and a negative ion emission probe which are arranged in pairs and symmetrically, and a distance between the positive ion emission probe and the negative ion emission probe is not less than 50 mm.

9. The plasma module according to claim 8, wherein
there is one or more groups of positive ion emission probes and negative ion emission probes arranged in pairs and symmetrically, and when there are a plurality of groups of positive ion emission probes and negative ion emission probes arranged in pairs and symmetrically, the positive ion emission probes and/or negative ion emission probes of different groups of the plurality of groups are spaced apart from each other,
a distance between the positive ion emission probe and the negative ion emission probe in different groups of the plurality of groups is greater than 50 mm; and/or
a distance between the positive ion emission probe and the negative ion emission probe in different groups of the plurality of groups is greater than a distance between the positive ion emission probe and the negative ion emission probe in the same group of the plurality of groups.

10. The plasma module according to claim 1, further comprising a mounting flange, wherein the mounting flange is integrally formed on the domed surface, one end surface of the mounting flange is coplanar with the flat surface, and the flat surface serves as a mounting surface, one or more mounting flanges are arranged, and when a plurality of mounting flanges are arranged, the plurality of mounting flanges are arranged at equal intervals in a circumferential direction of the flat surface.

11. The plasma module according to claim 10, wherein the mounting flange is provided with a mounting groove, and the plasma module further comprises a magnet, the magnet being arranged inside the mounting groove, the mounting groove and the magnet are clamped or bonded.

12. The plasma module according to claim 11, wherein the mounting flange is provided with a mounting hole, the magnet is of an annular structure, the magnet is provided with a center hole coaxial with the mounting hole, a aperture of the center hole is not less than that of the mounting hole, and the center hole and the mounting hole are configured to enable a fastener to pass through.

13. The plasma module according to claim 1, wherein a mounting pipe is arranged on the domed surface, the mounting pipe is configured to mount the plasma emission probe, and one end of the plasma emission probe protrudes from the inside of the plasma module through the mounting pipe to the outside of the domed surface, the mounting pipe protrudes from the domed surface in a direction away from the domed surface, and an inner wall surface of one end, away from the domed surface, of the mounting pipe abuts against an outer cladding layer of the plasma emission probe.

14. The plasma module according to claim 13, wherein
an inner diameter of the mounting pipe decreases gradually in a direction away from the domed surface, and a necking channel is formed in the mounting pipe; and/or
the mounting pipe is a telescopic pipe; and/or
the mounting pipe is a flexible pipe to facilitate adjustment of an angle of the mounting pipe with respect to the domed surface;
and/or an outer wall surface of the mounting pipe and the domed surface are integrally formed.

15. The plasma module according to claim 13, wherein the mounting pipe is angularly adjustably connected to the domed surface, the mounting pipe comprises a joint section and a mounting section which are connected in sequence, the joint section and the mounting section together forming a channel configured to enable the plasma emission probe to pass through, and
the joint section being pivotally connected to the domed surface; and/or
the joint section is pivotally connected to the mounting section.

16. The plasma module according to claim 1, wherein the plasma module further includes an indicator lamp and a circuit board, the indicator lamp is mounted on the domed surface, and both the indicator lamp and the plasma generator are electrically connected to the circuit board, and the indicator lamp is configured to indicate an operating state of the plasma generator, wherein
a distance between the indicator lamp and the edge of the domed surface is equal to the distance between the plasma emission probe and the edge of the domed surface; or
a distance between the indicator lamp and the edge of the domed surface is less than the distance between the plasma emission probe and the edge of the domed surface; or
a distance between the indicator lamp and the edge of the domed surface is greater than the distance between the plasma emission probe and the edge of the domed surface.

17. A disinfection and sterilization apparatus, comprising the plasma module according to claim 1.

18. An air duct assembly, comprising the plasma module according to claim 1.

19. The air duct assembly according to claim 18, wherein the air duct assembly comprises an air duct, the plasma module is arranged on the air duct, an angle β is formed between a direction of air flow of the air duct and a protruding direction of the plasma emission probe,
a scope of the angle β meets: 95°≤β≤120°; or
a scope of the angle β meets: 60°≤β≤85°.

20. The air duct assembly according to claim 19, wherein
the scope of the angle β meets: 95°≤β≤100°; or
the scope of the angle β meets: 105°≤β≤120°; or
the scope of the angle β meets: 60°≤β≤75°; or
the scope of the angle β meets: 80°≤β≤85°.
